(19) United States
(12) Patent Application Publication
(10) Pub. No.: US 2004/0034329 A1
(43) Pub. Date: Feb. 19, 2004

(54) Devices and methods for infusing a liquid into a catheter

(76) Inventors: Rita A Mankus, Lafayette, IN (US); Stephen R Ash, Lafayette, IN (US)

Correspondence Address: Gregory B Coy, Woodard Emhardt Naughton Moriarty & McNett, Bank One Center/Tower, Suite 3700, 111 Monument Circle, Indianapolis, IN, 46204-5137, US

(21) Appl. No.: 10/332,782

(22) PCT Filed: Jul. 13, 2001
(86) PCT No.: PCT/US01/41362

(51) Int. Cl. A61M 5/00; A61B 19/00; A61M 31/00; A61M 25/00

(52) U.S. Cl. 604/500; 604/523; 604/181; 604/403

(57) ABSTRACT

This invention generally relates to devices (1), methods and kits for use in connection with catheters, and more particularly to devices, methods and kits for infusing a liquid into a catheter, such as, for example, transcutaneous body access catheter. In one aspect, the invention involves infusing a lock solution into an indwelling catheter for preventing occlusion of the catheter and for inhibiting infection.

### REFERENCE TO RELATED APPLICATIONS

This application claims the benefit of U.S. Provisional Application No. 60/217,939, filed Jul. 13, 2001 and entitled DEVICES AND METHODS FOR LOCKING AN INDWELLING CATHETER, which is hereby incorporated by reference herein in its entirety.

### BACKGROUND OF THE INVENTION

This invention generally relates to devices, methods and kits for use in connection with catheters, and more particularly to devices, methods and kits for infusing a liquid into a catheter, such as, for example, a transcutaneous body access catheter. In one aspect, the invention involves infusing a lock solution into an indwelling catheter for preventing occlusion of the catheter and for inhibiting infection. In another aspect, the invention involves infusing a saline solution into an indwelling catheter to flush the contents of the catheter from the distal end of the catheter. The invention is particularly useful in connection with intravascular infusion catheters and systems, and can also be used in connection with peritoneal dialysis catheters, chest tubes, urinary catheters and the like.

By way of background, catheters are widely used to treat patients requiring a variety of medical procedures. Catheters can either be acute, or temporary, for short-term use or chronic for long-term treatment. Catheters are commonly inserted into central veins (such as the vena cava) from peripheral vein sites to provide access to a patient's vascular system. Catheters offer many advantages for patients; for example, chronic catheters provide ready access without repeated punctures or repeated vessel cannulation for administration of large volumes of fluids, nutrients and medications and for withdrawal of blood on an intermittent basis. With respect to the use of catheters for infusion of fluids, examples include the infusion of drugs, electrolytes or fluids used in chemotherapy. In chemotherapy, catheters are used for infusion of drugs on an intermittent basis, ranging from daily to weekly. Another example includes the use of catheters in hyperalimentation treatment, wherein the catheters are usually used for infusion of large volumes of fluids.

For hemodialysis, catheters are commonly used—usually three times per week—for aspiration of blood for dialysis treatment and rapid return of the blood to circulation after treatment. Although a preferred mode of vascular access for a hemodialysis patient involves using an arteriovenous (AV) fistula of either the upper or lower extremities or an arteriovenous “bridge” graft (typically utilizing PTFE), use of these access devices is not always possible or desirable. When either of these modes of vascular access is not available, for example, due to a paucity of adequate blood vessels for creation of AV “shunts” or due to nonoptimally functioning established AV shunts, a large bore venous line catheter is typically required for hemodialysis. Catheters used for hemodialysis usually include two relatively large diameter lumens (usually molded as one catheter) for aspiration and rapid return of blood required during the hemodialysis procedure. One lumen of such a catheter is used for aspiration, or removal, of blood, while the other lumen is used for returning the blood to the patient's bloodstream.

Catheter connections, such as, for example, connections of catheters to dialysis machine tubing, to IV line tubing, to infusion ports and to catheter caps, which are used to seal the end of a catheter to protect the sterility of the catheter and prevent fluid loss and/or particle contamination, are most often made utilizing the medical industry's standardized Luer taper fittings. These fittings, which may either be male couplings or female couplings, include a tapered end of standardized dimensions. Coupling is made by the press-fit of mating parts. A threaded lock-fit or other type of securing mechanism is commonly utilized to ensure the integrity of the pressure fit of the Luer fittings.

Catheters, especially chronic venous catheters, have drawbacks. One significant drawback is that such catheters can become occluded by a thrombus. In order to prevent clotting of catheters in blood vessels between uses, such as, for example, between dialysis treatments when the catheter is essentially nonfunctioning and dwells inside a “central” vein (i.e. superior vena cava, inferior vena cava, iliac, etc), the lumens of the catheter are often filled with a lock solution that comprises a concentrated solution of the commonly used anticoagulant, heparin (up to 10,000 units of heparin per catheter lumen). As used herein, the term “lock solution” refers to a solution that is injected or otherwise infused into a lumen of a catheter with the intention of allowing a substantial portion of the lock solution to remain in the lumen and not in the systemic blood circulation until it is desired or required to access that particular lumen again, typically for additional treatment, i.e., infusion or withdrawal of fluid. In addition, attention has been given to the development of alternative lock solutions with the goal of improving the patency rates of vascular catheters. Preferably the lock solution can remain in the lumen for a desired amount of time lasting from about 1 hour to 3 or 4 days or longer.

To lock an indwelling dialysis catheter, each lumen of the catheter can be filled with a lock solution including an anticoagulant, usually heparin, immediately after each use, and the anticoagulant theoretically remains within the catheter until the catheter is accessed again. The lock solution must be withdrawn from the catheter via aspiration prior to the next use of the catheter, such as, for example, prior to the next dialysis session, to prevent infusion of large quantities of an anticoagulant into a patient. Because of the large luminal diameters of hemodialysis catheters in general and the need to prevent intraluminal clotting, the concentration of the anticoagulant is generally much higher than what would be administered systemically. Therefore, great care must be taken to minimize the amount of the anticoagulant that passes into the patient's bloodstream because infusing even small amounts of an anticoagulant at such high concentrations in a patient might result in excessive bleeding. If a great excess of an anticoagulant is injected into the patient's blood during a catheter lock procedure or by mistaking an anticoagulant for some other fluid (such as saline) that is injected during the dialysis procedure, then harm can come to the patient. For example, excess heparin can cause excess anticoagulation of the patient's blood and bleeding from a number of sites.

Therefore, during a catheter lock procedure, it is important that the “lock” solution be carefully measured to avoid “anticoagulating” the patient, and the injected volume of solution is preferably exactly the same as the internal volume of the catheter to prevent clotting of the catheter lumen. Even when this volume is injected exactly, typically about ⅓ of the injected anticoagulant volume leaves the end of the catheter as a result of fluid mixing at the internal end of the catheter, causing some anticoagulation of the patient in the hours after a dialysis procedure.

Another significant drawback of indwelling intravascular catheters is that, even if extreme care is taken, the catheters can increase a patent's risk of infection. Great care must be taken in the placement and use of a chronic catheter to prevent infection of the patient at the site of access or within the vascular system. When catheters are inserted into veins or arteries, they bypass the protective dermis layer, and provide direct access to a patient's blood stream. This can, cause the inadvertent transfer of infectious agents into the vein or artery at the location of the catheter, either at the time of placement of the catheter, or over the course of its residence within a patient and across the patient's dermis layer. In addition, the foreign surfaces of catheters can create a smooth surface at which bacteria can grow, and at which the white cells are unable to surround or “phagocytize” the bacteria. Chronic venous catheters usually contain a DACRON cuff attached to the catheter and placed under the skin, which promotes ingrowth of fibrous tissue, fixes the catheter in position, and minimizes the occurrence of bacterial migration around the catheter from the external portion of the catheter to the systemic circulation.

Because such catheters are used to pass materials, such as dialysis blood, nutrients, medications and the like into a patient's bloodstream, it is readily understood that great care must be taken to ensure that the materials themselves that are being infused, and devices coming into contact with the catheter to infuse the materials, are aseptic. Catheters, particularly venous catheters, are frequently accessed with syringes, or uncapped and directly connected to IV lines.

This concern applies equally to lock solutions that are placed within catheters during periods of nonuse, and devices for introducing the lock solutions into the catheters. For example, heparin, when used as an anticoagulant in a catheter lock solution, has no anti-bacterial properties and, in fact, may promote growth of bacteria within the “biofilm” layer of protein on a catheter's surfaces (protamine has the opposite effect). The “biofilm” proteins on the catheter surfaces can protect bacteria from the bacteriocidal properties of antibiotics and white cells. If there is bacteremia (bacteria in blood), then the catheter surfaces within the vein or artery can become seeded with bacteria. In either case, the patient can develop septicemia (infection in the blood) and become seriously ill. Often these patients must be hospitalized and given intravenous antibiotics. In spite of this care, patients often remain seriously ill until the infected catheter is removed. Because catheters have a propensity to become contaminated, and because contamination can have dire consequences, great care must be taken to prevent the introduction of bacteria into a patient or into contact with an indwelling catheter.

For the reasons set forth above, significant care must be taken when infusing medications, nutrients and the like into a catheter, and when “locking” a catheter between uses, to minimize the risks associated with an indwelling catheter, including the risk of thrombosis or clotting, the risk of excessive anticoagulating and the risk of infection. Syringes are typically used to administer the required amount of catheter lock solution (determined by the catheter manufacturer) into an indwelling catheter after a given use. For example, at the end of the dialysis session, lock solutions are commonly introduced into both lumens of a dialysis catheter and eventually caps are placed on both the inflow and outflow catheter openings. The procedure of locking the catheter should be performed by aseptic technique but because of the need to “turn over” patients quickly in most dialysis units, techniques may become sloppy, thereby increasing the risk of infection. The aseptic techniques for the “take on” and “take off” of hemodialysis patients are often compromised, thereby resulting in the catheter related bacteremias/septicemias which is plaguing today's dialysis units, particularly since the number of catheter accesses have risen dramatically over the last several years. Over this same time period, the educational level of dialysis staff has generally declined due to lower salaries and the need to increase the already “tight” profit margins required by national dialysis chains.

In light of the above-described problems, there is a continuing need for advancements in catheter lock techniques, devices and procedures to improve the safety and efficacy of catheter locking procedures and of overall patient care. In particular, advancements are needed in locking techniques for indwelling dialysis catheters, including improved devices and techniques for reducing the incidence of infection or anticoagulation of a patient. Similarly, there is a continuing need for advancements in techniques, devices and procedures for infusing other types of liquids into transcutaneous catheters. The present invention is such an advancement and provides a wide variety of benefits and advantages.

### SUMMARY OF THE INVENTION

In one form of the invention, there is provided a device for use in “locking” an indwelling catheter between uses or for infusing a predetermined volume of another type of liquid into a catheter. The device has a connector having a suitable configuration for connection to the external end of an indwelling catheter, and includes a collapsible container configured to retain a lock solution and to dispense the solution into the catheter when the device is connected to a catheter and a medical provider or other user manually collapses the container.

Another form of the invention provides methods for locking an indwelling catheter between uses or for infusing a predetermined volume of another type of liquid into a catheter. The method includes affixing an inventive device, containing a predetermined volume of a lock solution, to the external end of an indwelling catheter by engaging the connector with the end of the catheter; and collapsing the container of the device to infuse the lock solution into the lumen of the catheter. Another embodiment further includes aspirating the lock solution from the catheter in preparation for a subsequent use of the catheter. In another form of the invention, an inventive device is used to infuse a saline flush solution into a catheter.

It is one object of the invention to provide a multifunctional cap that eliminates the need for precisely measuring and administering a catheter lock solution or other liquid via a syringe.

It is another object of the invention to provide techniques, devices and methods that lower the incidence of inappropriate (and even dangerous) anticoagulated events and the incidence of inappropriate underfilling of a catheter leading to clotting and subsequent inadequate dialysis treatments.

Another object of the invention is to provide techniques, devices and methods that lower the incidence of infection of a patient by contamination of a catheter during a catheter locking procedure or other liquid infusion procedure.

Further forms, embodiments, objects, features, and aspects of the present invention shall become apparent from description contained herein.

### BRIEF DESCRIPTION OF THE DRAWINGS

FIG. 1 is a schematic view of one embodiment of a device for infusing a lock solution into a catheter in accordance with the present invention, also showing a catheter.

FIG. 2 is a schematic view of another embodiment the invention.

FIG. 3 is a schematic view of another embodiment of a device in accordance with the present invention, wherein the container has a bulb configuration, the device schematically shown connected to a catheter.

FIG. 4 is a schematic perspective view of another embodiment of the invention, wherein the container has a straight bellows configuration.

FIG. 5 is a schematic perspective view of another embodiment of the invention, wherein the container has a fan-shaped bellows configuration, and the device is shown in an expanded orientation.

FIG. 6 is a schematic perspective view of the embodiment of FIG. 5, wherein the device is shown in a partially collapsed orientation.

FIG. 7 is a side view of another embodiment of the invention, wherein the container has a disk-shaped configuration.

FIG. 8 is an end view of the embodiment of FIG. 7.

FIG. 9 is a schematic view of another embodiment of the invention, also showing a catheter.

### DETAILED DESCRIPTION OF PREFERRED EMBODIMENTS

For the purposes of promoting an understanding of the principles of the invention, reference will now be made to the embodiments set forth herein and specific language will be used to describe the same. It will nevertheless be understood that no limitation of the scope of the invention is thereby intended. Any alterations and further modifications in the described processes, systems or devices, and any further applications of the principles of the invention as described herein, are contemplated as would normally occur to one skilled in the art to which the invention relates.

In order to prevent clotting of catheters between use, catheters are commonly filled with lock solutions including an anticoagulant agent and sometimes additional compositions. Inventive devices and methods for infusing a liquid into a catheter find one advantageous use in infusing a lock solution into an indwelling catheter, such as, for example, an indwelling transcutaneous hemodialysis catheter. For purposes of describing the invention, techniques, devices and methods for locking dialysis catheters are described in detail. It is not intended, however, that use of the invention be limited to locking dialysis catheters, it being contemplated that the invention also finds advantageous use in locking of a wide variety of other indwelling catheters. In addition, it is not intended that the invention be limited to infusing lock solutions, it being contemplated that the invention also finds advantageous use to infuse a wide variety of other liquids, such as, for example, nutrients, medicines, saline and the like, into a variety of catheters.

The filling, or “locking,” of a hemodialysis catheter after completion of a hemodialysis treatment typically involves a series of steps, many of which expose the lumen of the catheter, or instruments or fluids coming into contact with the catheter lumen, to possible infection. Steps of the procedure include, for example, clamping the catheter body; disconnecting the catheter's Luer connectors from dialysis machine blood lines; placement of a cap on the catheter; filling a syringe with a predetermined amount of a lock solution as prescribed by the catheter manufacturer; inserting the tip of the syringe needle through the cap and into the lumen of the catheter; unclamping the catheter body; infusing the lock solution into the catheter from the syringe; re-clamping the catheter body and withdrawing the needle from the catheter. Other procedures involve the placement of a cap having a “needleless port,” which are well known in the art, onto the end of the catheter. Procedures using such a cap also involve a series of steps in which the lumen of the catheter is exposed to possible infection. Another approach to “locking” a catheter after use is to clamp the catheter body; remove the fluid connecting tubings from the Luer connector; attach a syringe with the locking solution and configured for connection to the Luer connector; unclamp the catheter; infuse the locking solution; clamp the catheter; detach the syringe; and apply a “locking” or non-penetrable cap.

It is readily understood that these types of procedures have a number of disadvantages. For example, blood in the catheter begins to clot shortly after flow ceases at the end of a dialysis treatment, and any delay in the process may cause undesired thrombosis. Also, the procedure involves placement of a number of foreign objects and surfaces, including a catheter cap, a syringe needle, or the like, directly into contact with the lumen of the catheter. Each point of contact increases the risk of infecting the catheter with bacteria. These procedures require multiple instances during which the external end of the catheter is uncapped or otherwise exposed to possible infection.

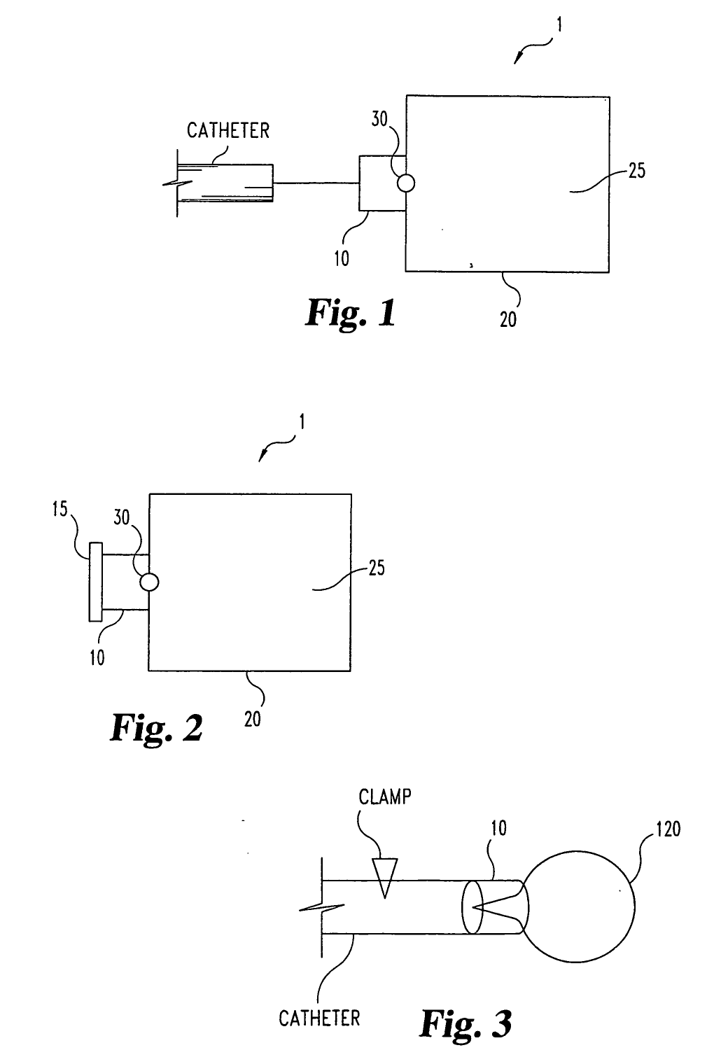

In one aspect, the present invention provides a pre-filled device for “locking” an indwelling catheter between uses. Use of an inventive infusion device can advantageously eliminate the need for a number of steps in a catheter locking procedure, can minimize the length of time that blood remains in the catheter lumen after a dialysis treatment is completed, can minimize the amount of time that the external end of a catheter must remain exposed, or uncapped, before and after a dialysis procedure is performed, and reduces the number of foreign objects that come into contact with the lumen of the catheter. One embodiment of the invention is depicted schematically in FIG. 1, wherein device 1 includes collapsible container 20 configured to retain a lock solution or other liquid, and connector 10. Connector 10 is configured for connection to the proximal (external) end of an indwelling catheter in a manner whereby the solution can be dispensed from the container into the catheter when a medical provider or other user manually collapses the container.

Most conventional catheters are configured to receive caps or other connections in a relationship commonly referred to as a Luer lock connection. In one embodiment of the invention, connector 10 has a Luer locking configuration for attachment to most conventional catheters. It is understood that the connector can have alternate configurations as would be apparent to a person of ordinary skill in the art, for example to engage a catheter having a different design or mode of attachment.

Collapsible container 20 defines cavity 25 and port 30, such as, for example, an orifice or a valve, through which the cavity fluidly communicates with the exterior of the container. It is, of course, understood that in embodiments featuring a valve, the cavity fluidly communicates with the exterior of the container only when the valve is open. When the valve is closed, the cavity is sealed and does not fluidly communicate with the exterior of the container.

Connector 10 is affixed to container 20 in a relationship whereby connector 10 overlays port 30. In other words, connector 10 is attached to container 20 about the port to provide an arrangement in which connector 10, when mated to the proximal end of a catheter, provides a sealed connection in which cavity 25 fluidly communicates with the lumen of the catheter through port 30. When device 1 is connected to a catheter by engagement of connector 10 to the proximal (external) end of the catheter, an arrangement is provided whereby port 30 is disposed between cavity 25 and the lumen of the catheter, thereby defining a closed system including cavity 25, port 30, the lumen of the catheter, and the patient's vascular system.

In one embodiment, the connector is configured for connection to a catheter having a fitting on its proximal end, which fitting is configured to mate with the connector. In one embodiment, the fitting is one of a male Luer coupling or a female Luer coupling, and the connector comprises the other of a male Luer coupling or a female Luer coupling. In this embodiment, the connector is affixed to the catheter by press-fitting the fitting and the connector. In another embodiment, the connector includes a fastener to maintain integrity between the fitting and the connector. A wide variety of fasteners are available for such use, as would occur to a person of ordinary skill in the art. In one embodiment, the proximal end of the catheter defines a flange or a screw thread, and the fastener defines a flange or a screw thread configured to cooperate with the flange or screw thread of the catheter to maintain integrity between the fitting and the connector.

In one embodiment of the invention, port 30 is a valve. A wide variety of valves can be used in accordance with the invention as would occur to a person of ordinary skill in the art. In one embodiment, the valve is a one-way valve such as, for example, a “duck-bill valve.” The duck-bill valve is preferably configured such that, in the absence of external actuation, fluid flows through the valve in a first direction in response to a pressure gradient across the valve, but is prevented from flowing through the valve in the opposite direction. Preferably, a moderate threshold pressure is required to cause the fluid to flow in the first direction. In another embodiment, the valve is configured such that it can be manually actuated to open the valve and allow fluid flow in either direction, the direction of flow depending upon the pressure gradient across the valve. In another embodiment, port 30 is a stopcock valve, a variety of which are readily available commercially. Duck-bill valves, manually actuated valves, stopcock valves and a wide variety of valves having other configurations are commonly known to a person of ordinary skill in the art and readily available commercially.

By way of example, in an embodiment utilizing a duck-bill valve, the solution is held within the cavity until pressure in the cavity resulting, for example, from a medical provider manually collapsing the container, causes fluid to flow in the first direction from the container through the valve. As such, once the device of this embodiment is connected to a catheter, the container can be manually collapsed to cause the lock solution to enter the catheter through the valve. Once the lock solution has entered the catheter, the one-way valve prevents flow in the second direction to ensure that the solution is not prematurely withdrawn from the catheter.

This embodiment of the invention is particularly useful when the catheter being locked does not include a clamp at or near the external end of the catheter. Although most catheters used for dialysis include such a clamp, some do not, and for catheters without clamps, this type of valve is useful for preventing flow of fluids out of the catheter during the lock period (i.e., period of nonuse).

In another embodiment of the invention, port 30 is an orifice. In the device of this embodiment, the orifice preferably has a size whereby the surface tension of the lock solution is great enough to keep the fluid in the container during normal handling and until pressure is applied to the fluid. In this regard, lock solutions have been proposed that include viscosifying agents to increase the viscosity of the solution. Examples of such compositions are described in International Application No. PCT/US99/19307 entitled METHOD OF ENHANCING CATHETER PATENCY USING A CITRATE SALT CATHETER LOCK SOLUTION (International Publication No. WO 00/10385), in U.S. Provisional Patent Application No. 60/203,358 entitled A CATHETER LOCK SOLUTION INCLUDING A PHOTO-OXIDANT, filed May 10, 2000, and in International Application No. PCT/US01/15177 entitled A CATHETER LOCK SOLUTION INCLUDING A PHOTO-OXIDANT, filed May 10, 2001, each of which is hereby incorporated herein by reference in its entirety. Viscosifying agents are included in such lock solutions so that the solutions have viscosities sufficiently high to minimize displacement of the lock solution with blood in an indwelling intravascular catheter over time so that a large proportion of the lock solution remains in the catheter between uses. Other lock solutions that can be used in connection with the present invention have much lower viscosities. In this regard, it is understood that the desired size of the orifice in an inventive device may depend upon the viscosity of the lock solution therein.

In a preferred embodiment, the orifice has a generally circular shape and has a diameter of from about 0.25 mm to about 2 mm. In another embodiment, the orifice has a diameter of from about 0.75 mm to about 1 mm. In another embodiment, the orifice has a diameter of about 0.5 mm. It is understood that an embodiment having an orifice will most commonly be used with a catheter that has a clamp or other device at or near its external end for preventing flow of fluids into and out of the catheter after the lock solution has been infused.

In addition, the device can also include a plug 15 configured for connection to the connector 10 in a manner similar to the external end of the catheter. An embodiment schematically depicting plug 15 is set forth in FIG. 2. Plug 15 can have a wide variety of configuration as would occur to a person of ordinary skill in the art, and can resemble a catheter cap, provided that the cap has a fitting configured to mate with the connector. Plug 15 can advantageously be connected to a filled device to prevent the lock solution from leaking from the container during shipment, storage, handling and the like. The presence of a plug is particularly useful in embodiments having an orifice rather than a valve. Just before the device is used to lock a catheter, the plug 15 can be disconnected, thereby freeing the device for connection to the catheter. Such a plug can also be used to advantage in a device having a valve as described above.

It is readily understood that container 20 can have a wide variety of sizes, shapes and configurations. For example, indwelling catheters can have lumens of widely varying volumes, most dialysis catheters having lumen volumes of between about 1.4 cc and about 2.1 cc, and most chronic venous catheters having lumen volumes of from about 0.5 cc to about 1.5 cc. In one embodiment, an inventive locking device used to lock a given catheter has an internal volume that corresponds to the lumen volume of the catheter being locked. Use of the phrase “corresponds to” is intended to refer to a volume that a medical provider would select to lock a catheter of a given volume between uses. It is generally understood that the volume of lock solution introduced into a catheter is preferably from about 80% to about 100% of the volume of the catheter lumen. An advantage of the present invention is that an inventive device can be manufactured to correspond to catheters of specific volumes, and the need for careful measurement of a lock solution at the time of the locking procedure, and the risk of mistake attendant thereto, is thereby eliminated.

In another embodiment, an inventive device has an internal volume greater than the internal volume of the catheter to be locked. It is understood that, in certain embodiments, the container is not filled to capacity prior to use, but rather contains a predetermined amount of a lock solution that corresponds to the catheter. The container preferably does not contain air or other gas, and thus is prepared to have an initial, partially collapsed arrangement. In this regard, it is a common practice when aspirating a locked catheter to aspirate a larger volume than the internal volume of the catheter, thereby ensuring that substantially all of the lock solution has been recovered. A device of this embodiment can advantageously be used to aspirate such a larger volume into the container at the end of a lock period without the necessity of disconnecting the device or accessing the container with a needle or needleless syringe. In one embodiment, the device has an internal volume of from about 0.5 to about 3.5 cc. In another embodiment, the device has an internal volume of from about 1 to about 3 cc. In still another embodiment, the device has an internal volume of about 2.5 cc.

In addition to variations in size, inventive devices can have a wide variety of configurations. In one embodiment of the invention, depicted schematically in FIG. 3, the container is a shape-memory collapsible bulb 120 configured to return to its original shape when external forces are removed. An advantage of such a shape-memory container is that, after a catheter is locked by collapsing the container, and thereby introducing the lock solution into the catheter, the container creates a negative pressure, which can be used to pull the solution back into the container at the end of a lock period when the catheter is being prepared for a subsequent use. It is, of course, understood that, when such a shape-memory container is used, the device or the catheter being locked must include a clamp, valve or other device to prevent premature withdrawal of the lock solution from the catheter.

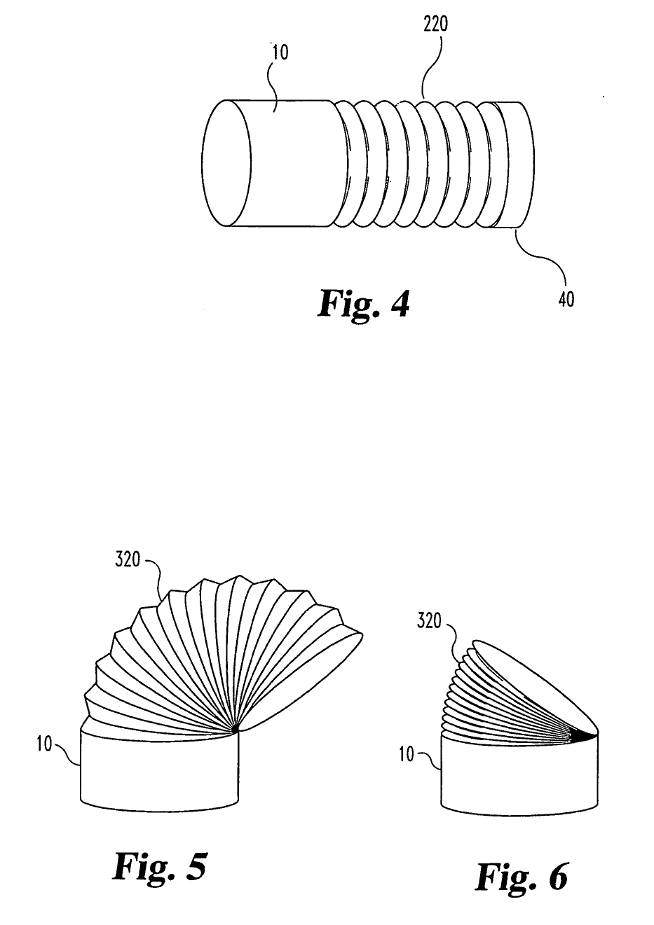

In another embodiment of the invention, depicted in FIG. 4, the container is a flexible and expansible accordion vessel 220 having a configuration of a bellows. Thus, in this embodiment, by contraction of the bellows, the container passes the lock solution through the port, and by expansion of the bellows, the container draws the lock solution back through the port. The configuration of container 220 in this embodiment is referred to herein as a “straight bellows.”

As depicted in FIG. 4, the container can also include a cap 40 configured to engage the connector 10 when the device is in the contracted state (i.e., after the lock solution is introduced into a catheter). An advantage of this configuration is that, once the lock solution is introduced into the lumen, the device is compressed to a small size, and is not bulky. This configuration is advantageous because the device can remain connected to the catheter for an extended period of time. Because of the collapsing nature of the device, devices in accordance with this embodiment can be made to have sizes similar to that of conventional locking caps.

In another embodiment, depicted in FIGS. 5 and 6, container 320 has a flexible and expansible fan-shaped bellows configuration. In this embodiment, contraction of the bellows (i.e., “closing the fan”) causes the container to pass the lock solution through the port and into the catheter lumen (when the device is connected to a catheter), and expansion of the bellows causes a pressure gradient across the port, which can be used to draw the lock solution back into the container through the port. In this embodiment, the bellows portion can be configured to hinge about a fixed point adjacent connector 10. This embodiment is schematically represented in FIG. 5 in an expanded state, and schematically represented in FIG. 6 in a partially contracted state.

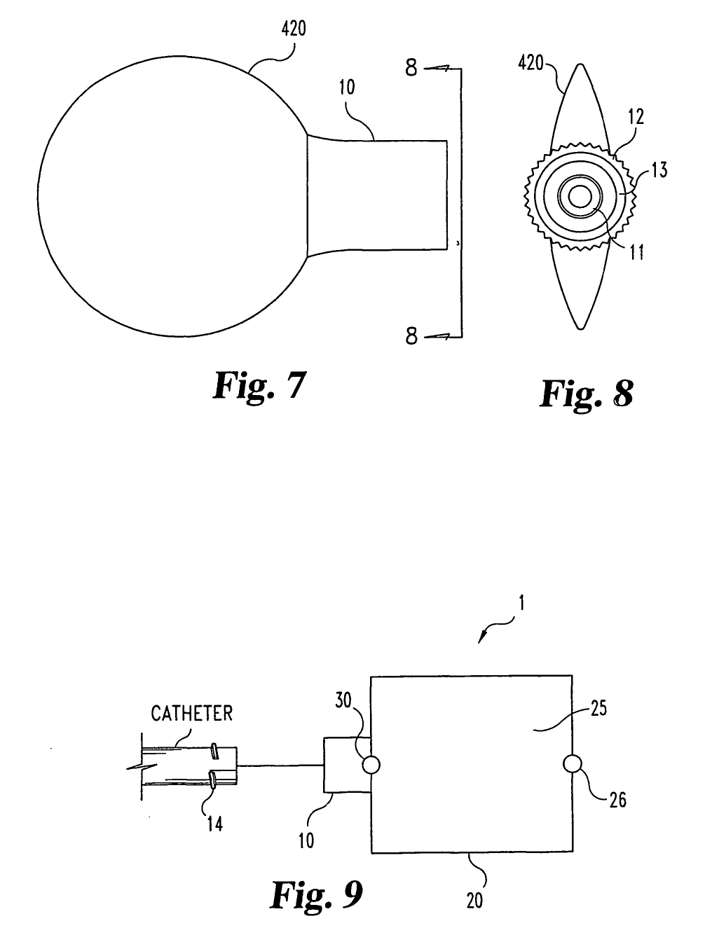

In another embodiment, depicted in FIGS. 7 and 8, container 420 has a flexible disk-shaped configuration. FIG. 8 sets forth an end view of this embodiment, and depicts a Luer lock configuration that includes screw-threaded fastener 12 featuring thread 13 configured to engage one or more flanges or screw threads on the proximal end of a catheter (or one or more flanges or screw threads on a plug, when present). For purposes of example, flange 14 depicted on the catheter represented in FIG. 9 is configured to receive thread 13 to hold connector 10 to the catheter, thereby maintaining the integrity of the Luer connection. Connector 10 also includes male Luer coupling 11 configured to mate with a corresponding female Luer coupling of a catheter. It is of course understood that in alternate embodiments, male coupling 11 could be substituted with a female coupling or other matable element, and screw-threaded fastener 12 could be substituted for an alternate fastening mechanism, to provide an alternate device configured for connection to catheters having various configurations. Such substitutions are well within the purview of a person of ordinary skill in the art.

Inventive devices can be prepared from a variety of materials, including, for example and without limitation, silicon, polyurethane, polyvinyl, silicone, or silastic elastomer. While it is desirable that the connector be made of a generally rigid material, such as, for example, polycarbonate, polypropylene or high density polyurethanethe, the container is preferably made of a flexible polymeric composition, such as, for example, polyvinylchloride (PVC) or low density polyurethane. In a preferred aspect of the invention, inventive devices are prepared from a polycarbonate connector and PVC container. The device can be made by blow molding using a process whereby the selected polymer in the container portion of the device is formed into a thin, flexible layer and the polymer in the connector portion of the device is formed into thicker, more rigid layers. In addition, a movable fastener element, such as a threaded screw-on lock for a Luer lock connector, can be made as a separate molded piece, perhaps of polycarbonate, for example, and formed to snap into place with the connector.

In one embodiment, the composition of container 20, 120, 220, 320, 420 is transparent or translucent, thereby allowing the user to determine the color of a fluid in the container. For example, a lock solution used in connection with the device can have an inherent color, as described in U.S. Provisional Patent Application No. 60/203,358 entitled A CATHETER LOCK SOLUTION INCLUDING A PHOTO-OXIDANT, filed May 10, 2000, and in International Application No. PCT/US01/15177 entitled A CATHETER LOCK SOLUTION INCLUDING A PHOTO-OXIDANT, filed May 10, 2001, each of which is hereby incorporated herein by reference in its entirety. Use of a transparent or translucent material for container 20 allows a medical provider or other user to determine if such a solution is contained in the device simply by viewing the solution through the container. In addition, an inventive device can be used to aspirate a lock solution from a catheter prior to a subsequent use of the catheter or infusion of a fresh lock solution, as described more fully below. The transparency or translucency of the container will allow a user to determine if blood has entered the container during aspiration of the lock solution.

In certain embodiments of the invention, device 1 is pre-filled, and therefore features a liquid, such as, for example a lock solution, contained in cavity 25. A lock solution used to lock a hemodialysis catheter can have a wide variety of formulations, many of which are available commercially. The solution preferably includes an anticoagulant, many of which are well known to those skilled in the art, including, for example and without limitation, citrate, heparin, urokinase, tissue plasminogen activation (tPA) and mixtures of these agents. As described in pending U.S. Provisional Patent Application No. 60/203,358 entitled A CATHETER LOCK SOLUTION INCLUDING A PHOTO-OXIDANT, filed May 10, 2000, and in International Application No. PCT/US01/15177 entitled A CATHETER LOCK SOLUTION INCLUDING A PHOTO-OXIDANT, filed May 10, 2001, the lock solution can also include a photo-oxidant. The solution can also include a variety of additional materials. For example, in certain preferred embodiments, the lock solution also includes an antibacterial or antimicrobial agent. Such antibacterial and antimicrobial agents are well known to those skilled in the art and can include, for example and without limitation, gentamicin, vancomycin, and mixtures of these agents.

The viscosity of the lock solution can be varied by including a viscosifying agent. In certain embodiments of the invention, therefore, the lock solution also includes a viscosifying agent. It is well known that catheters are manufactured to have a variety of configurations and lumen diameters. For example, catheters can include single or double lumens. The double lumens can be fused adjacent to each other or they can be concentric. The lumens can have varying cross-sectional areas and shapes, ranging from substantially circular to substantially ovoid. A phenomenon common to most lock solutions, as discussed above, is that a portion of the solution at the distal end of the lumen diffuses into the patient's blood stream and is replaced in the catheter by blood. While not intending to be bound by any theory, it is thought that the rate of diffusion of a lock solution from a lumen can be influenced by the cross-sectional shape and area of the particular lumen(s), the density of the lock solution, and the viscosity of the lock solution. Typically, high-density lock solutions such as those of relatively high concentrations of citrate tend to fall out of the lumen of the catheter, allowing blood to enter into the lumen.

A lock solution that can advantageously be used in connection with the present invention can be prepared to have a viscosity and density such that a substantial portion of the lock solution does not diffuse or flow out of a catheter lumen within several days. Viscosifying agents useful with the present invention include those pharmaceutically acceptable agents known or commonly used in treatment of animals including humans. Examples include, but are not limited to, dextran, polyethylene glycol, glycerin, polygeline, and non-metabolizable sugars such as sorbitol and mannitol and mixtures of these compounds. While it is understood that optimal viscosity and density are dependent upon the shape and size of a particular lumen, a person of ordinary skill in the art, in view of the description herein, can readily determine a desired density and viscosity for a particular catheter without undue experimentation. It is, of course, also understood that the viscosity of the lock solution used in connection with an inventive device having an orifice port should be considered in determining the preferred size of the orifice, as described above. It is well within the purview of a person of ordinary skill in the art to determine the proper size for a given use.

A lock solution can also be prepared to include a variety of other pharmaceutically acceptable agents. For example, the lock solution can include salts, such as, for example, sodium chloride or other sodium salts. By “pharmaceutically acceptable”, it is meant that the lock solution and the included salts and other additives which are, within the scope of sound medical judgment, suitable for use in contact with tissues of humans and lower animals without undue toxicity, irritation, allergic response, and the like, and are commensurate with the reasonable benefit/risk ratio. For example, pharmaceutically acceptable salts are well-known in the art, for example, as found in S. M. Berge et al. described in detail in J. Pharmaceutical Science, 66:1-19, 1977.

Examples of excellent lock solutions for use in accordance with the invention are described in International Application No. PCT/US99/19307 entitled METHOD OF ENHANCING CATHETER PATENCY USING A CITRATE SALT CATHETER LOCK SOLUTION (International Publication No. WO 00/10385), and in U.S. Provisional Patent Application No. 60/203,358 entitled A CATHETER LOCK SOLUTION INCLUDING A PHOTO-OXIDANT, filed May 10, 2000, and in International Application No. PCT/US01/15177 entitled A CATHETER LOCK SOLUTION INCLUDING A PHOTO-OXIDANT, filed May 10, 2001, each of which is hereby incorporated herein by reference in its entirety.

To prepare device 1 for use, a predetermined amount of a lock solution is placed within the cavity 25 in a manner whereby the solution is held within the cavity 25 until an external force collapses the container 20, thereby pushing the solution out of the cavity 25 through the port 30. In one manner of placing the solution into the cavity, a needle is inserted into the cavity and a predetermined amount of the solution is metered into the cavity through the needle. If this manner of filling is used, it is understood that the container can include an injection port 26, as depicted schematically in FIG. 9, such as, for example, a “self-healing” port that is configured to become resealed after a needle is withdrawn therefrom. The injection port can comprise, for example, a rubber septum, well known to a person of ordinary skill in the art. If the body of the container is made of a composition capable of resealing upon withdrawal of a needle, such an injection port is not necessary. Alternatively, an injection port 26 can simply be formed as a thickened area of the container for increased durability. As a further alternative, the injection port can be a needleless port, as are well known in the art, for receiving a needless syringe to provide fluid communication with the cavity. It is readily understood that injection ports having a wide variety of configurations can be placed at a wide variety of locations on the container. In one embodiment, as depicted in FIG. 9, the injection port 26 is positioned substantially opposite port 30. It is understood that the cavity can be filled manually, for example, using a needle and syringe or needleless syringe at the site of the device's intended use, or can be accomplished at a manufacturing facility using a machine designed to meter the solution into such devices.

In another manner of introducing the solution into the device, the solution is metered into the cavity through port 30. Port 30 can also be used in accordance with the invention to purge the container of air, if any is present in the cavity. It is of course understood that the solution is preferably introduced into the cavity using aseptic technique. The invention also contemplates manufacturing protocols in which a device, after the solution is introduced thereinto, is sterilized using, for example, an autoclave procedure, to ensure that the device is free from bacteria.

In an embodiment utilizing a one-way valve, the solution is held within the cavity until pressure in the cavity resulting, for example, from a medical provider manually collapsing the container, causes fluid to flow in the first direction from the container through the valve. As such, once the device of this embodiment is connected to a catheter, the container can be manually collapsed to cause the lock solution to enter the catheter through the valve. Once the lock solution has entered the catheter, the one-way valve prevents flow in the second direction to ensure that the solution is not prematurely withdrawn from the catheter.

This embodiment of the invention is particularly useful when the catheter being locked does not include a clamp at or near the external end of the catheter. Although most catheters used for dialysis include such a clamp, some do not, and for catheters without clamps, the valve is useful for preventing flow of fluids out of the catheter during the lock period (i.e., period of nonuse).

When it is desired to again use the catheter, the device of this embodiment can be used to aspirate the catheter by manually actuating the valve to allow fluid flow in either direction. When the valve is actuated, the blood pressure in the accessed vein will cause the fluid in the catheter to pass into the container through the valve. It is understood that, for catheters used to access other body cavities, such pressure may not be present, in which case it may be necessary to create a negative pressure in the container. This may be accomplished using a syringe, as described more fully below.

To lock an indwelling catheter, the external end of a catheter to be locked is disconnected from any caps or conduits that may have been attached thereto during a treatment, and an inventive device, containing a predetermined volume of a lock solution, is affixed thereto by engaging the connector with the end of the catheter. Once engaged, the catheter and the device are in a relationship whereby the port of the device is adjacent the lumen of the catheter. For example, in an embodiment wherein the port is an orifice, the catheter and the device are in a relationship whereby the cavity of the container is in fluid communication with the lumen of the catheter. In this embodiment, the medical provider causes the container to collapse, thereby causing the lock solution to enter the lumen of the catheter, while simultaneously pushing the contents of the lumen into the patient's bloodstream.

The container is kept in the collapsed position, for example, by clamping the catheter or by closing the valve, if present, until the time that the catheter is to be used again, at which time the lock solution is withdrawn from the catheter for disposal. Withdrawal of the lock solution from the catheter can be accomplished in certain aspects of the invention by causing the container to revert to its original shape, thereby aspirating the lock solution back into the container. Alternatively, if a device is used having a container that includes an injection port, as described above, a syringe or a needle and syringe can be used to aspirate the lock solution by connecting the syringe to the injection port or by inserting the needle into the container through the injection port and drawing the lock solution through the injection port and into the syringe.

When using a device that includes a manual valve, of course, the manner of using the device differs in that, before causing the container to collapse, it is necessary to open the valve. In this embodiment, the medical provider opens the valve and then causes the container to collapse, thereby causing the lock solution to enter the lumen of the catheter, while simultaneously pushing the contents of the lumen into the patient's bloodstream. The container can be kept in the collapsed position in this embodiment by simply closing the valve. When the catheter is to be used again, withdrawal of the lock solution from the catheter can be accomplished by opening the valve and then either causing the container to revert to its original shape or withdrawing the solution using a syringe or a needle and syringe as described above.

Once a lock solution is infused into the lumen of the catheter, it is allowed to remain until that particular catheter or lumen is desired to be accessed again. Especially with heparin, it is important to remove the catheter lock before starting the dialysis procedure, or using the catheter for fluid infusion. A great advantage of a lock solution including methylene blue or other photo-oxidant or colorant, as described above, is that it provides color to the lock solution. This color will indicate to health professionals using the device or that it or a catheter is filled with an anticoagulant.

Devices in accordance with various embodiments of the invention have dual functionality—first as a way to administer a catheter lock solution with precision and efficiency, and second as a catheter cap. Use of an inventive device eliminates the need for a prefilled syringe or for carefully measuring and filling a syringe with a lock solution, as well as eliminate at least one step in the “take on” and “take off” of dialysis patients. Since the actual pathogenesis of catheter related bacteremia is not known but may be in part be due to colonization of ends of the catheter limbs, use of an inventive device containing a lock solution with both anticoagulant/antibacterial properties is expected to result in significant decreases in the occurrence of bacteremia. The device not only administers the anticoagulant/antibacterial lock solution but because of its contact with the solution, may also prevent colonization surrounding hemodialysis catheters. The device can also be used for acute catheters which have an even higher rate of catheter related bacteremias.

The invention is, of course, also contemplated for use in connection with catheters used to access other body cavities, and cathers used to access a patient's vascular system for purposes other than dialysis. For example, catheters are placed into arteries to measure blood pressure or remove arterial blood for analysis of gases reflecting lung function. Catheters are placed into the peritoneum (the space surrounded by the peritoneal membrane and external to organs in the abdomen) to perform peritoneal dialysis and remove fluids and toxins from the patient. Other catheters are placed into the fluid around the nervous system (cerebral spinal fluid) for removal of this fluid or administration of drugs, and into the subcutaneous space for administration of various drugs or fluids. Other catheters are placed in the bladder for an intermittent drainage of urine. Such catheters are also subject to infection and to other problems addressed herein, and an inventive device can advantageously be used to lock such catheters as well.

In another embodiment of the invention, an inventive device contains a saline solution and is used to infuse the saline solution into a catheter. This embodiment finds advantageous use, for example, when a catheter is used to deliver a bolus of medicine to a patient. It is readily understood that, when the medicine is introduced into the proximal end of a catheter, at least a portion of the dose remains in the catheter unless flushed out by infusion of a second fluid into the catheter. The invention therefore finds advantageous use as a saline flush delivery device, whereby a dose of medicine can be flushed through the catheter using an inventive device, and the device can remain affixed to the catheter as a cap to thereby reduce the exposure of the catheter lumen to infection.

As can be appreciated by those of skill in the art, in one embodiment there has been described a method for infusing a liquid into a catheter. The method includes: (1) providing a catheter defining lumen, a proximal end and a distal end, wherein the proximal end includes a fitting configured to mate with a connector; (2) providing a device including: (i) a collapsible container defining a cavity and defining a port; (ii) a liquid contained within the cavity, wherein the volume of the liquid has a predetermined ratio to the volume of the lumen of the catheter; and (iii) a connector attached to container about the port, wherein the connector is configured to mate with the fitting to provide a sealed connection in which the cavity fluidly communicates with the lumen through the port; (3) affixing the device to the catheter by mating the connector with the fitting; and (4) applying positive pressure to the liquid to cause the liquid to exit the cavity through the port and enter the lumen of the catheter at the proximal end.

In one embodiment, the ratio is from about 1:2 to about 2:1. In yet another embodiment, the ratio is from about 0.8:1 to about 1:0.8. In still another embodiment, the liquid has a volume of from about 1 to about 5 milliliters. In certain embodiments, the liquid is a catheter lock solution. In other embodiments, the liquid is a saline solution. In another embodiment, wherein the liquid is a saline solution, the method also includes, prior to said affixing, introducing into the catheter a liquid selected from the group consisting of a dose of medicine and a dose of nutrients. In still other embodiments, the liquid is selected from the group consisting of a nutrient solution and a medicine. In yet another embodiment, the cavity is essentially free from air. In certain embodiments, the catheter is a transcutaneous body catheter, wherein the distal end is internally positioned within a patient, and wherein the proximal end is external to the patient. In another embodiment, the catheter is a hemodialysis catheter.

In another embodiment of the invention, the method also includes: (1) allowing the device to remain affixed to the catheter until a hemodialysis treatment is indicated; (2) applying a negative pressure to the cavity to cause a predetermined volume of fluid to exit the lumen of the catheter at the proximal end and enter the cavity through the port; (3) disconnecting the device from the catheter; and (4) connecting one or more hemodialysis conduits to the catheter for the dialysis treatment.

In certain embodiments, the fitting is one of a male Luer coupling or a female Luer coupling, and the connector comprises the other of a male Luer coupling or a female Luer coupling. The connector is affixed to the fitting by press-fitting the fitting and the connector. In other embodiments, the connector includes a fastener to maintain integrity between the fitting and the connector. In certain embodiments, the proximal end of the catheter defines a first flange or a first screw thread, and the fastener defines a second flange or a second screw thread configured to cooperate with the first flange or first screw thread to maintain integrity between the fitting and the connector.

In various embodiments, the container comprises a disk shape, a straight bellows configuration, or a fan-shaped bellows configuration. In addition, in various embodiments, the port is a one-way valve, a duck-bill valve, a stopcock valve, a manually actuated valve, or an orifice. In certain embodiments, the orifice has a diameter of from about 0.25 to about 2 mm. In other embodiments, the orifice has a diameter of from about 0.75 to about 1 mm. In still other embodiments, the orifice has a diameter of about 0.5 mm.

In another aspect of the invention, there is provided a device for infusing a liquid into a catheter, wherein the catheter defines a lumen, a proximal end and a distal end, and wherein the proximal end includes a fitting configured to mate with a connector, the device. In one embodiment, the device includes: (1) a collapsible container defining a cavity and defining a port; (2) a liquid contained within the cavity, wherein the volume of the liquid has a predetermined ratio to the volume of the lumen of the catheter; and (3) a connector attached to container about the port. The connector is configured to mate with the fitting to provide a sealed connection in which the cavity fluidly communicates with the lumen through the port. The port is configured to allow the passage of the liquid from the cavity into the lumen of the catheter at the proximal end when a positive pressure is applied to the liquid.

In one embodiment, the ratio is from about 1:2 to about 2:1. In yet another embodiment, the ratio is from about 0.8:1 to about 1:0.8. In still another embodiment, the liquid has a volume of from about 1 to about 5 milliliters. In certain embodiments, the liquid is a catheter lock solution. In other embodiments, the liquid is a saline solution. In still other embodiments, the liquid is selected from the group consisting of a nutrient solution and a medicine. In yet another embodiment, the cavity is essentially free from air. In certain embodiments, the catheter is a transcutaneous body catheter, wherein the distal end is internally positioned within a patient, and wherein the proximal end is external to the patient. In another embodiment, the catheter is a hemodialysis catheter.

In certain embodiments, the fitting is one of a male Luer coupling or a female Luer coupling, and the connector comprises the other of a male Luer coupling or a female Luer coupling. The connector is affixed to the fitting by press-fitting the fitting and the connector. In other embodiments, the connector includes a fastener to maintain integrity between the fitting and the connector. In certain embodiments, the fastener defines a first flange or a first screw thread configured to cooperate with a corresponding second flange or second screw thread of a catheter to maintain integrity between the fitting and the connector.

In various embodiments, the container comprises a disk shape, a straight bellows configuration, or a fan-shaped bellows configuration. In addition, in various embodiments, the port is a one-way valve, a duck-bill valve, a stopcock valve, a manually actuated valve, or an orifice. In certain embodiments, the orifice has a diameter of from about 0.25 to about 2 mm. In other embodiments, the orifice has a diameter of from about 0.75 to about 1 mm. In still other embodiments, the orifice has a diameter of about 0.5 mm.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected.

What is claimed is:

1. A method for infusing a liquid into a catheter, comprising:
providing a catheter defining lumen, a proximal end and a distal end, wherein the proximal end includes a fitting configured to mate with a connector;
providing a device including:
a collapsible container defining a cavity and defining a port;
a liquid contained within the cavity, wherein the volume of the liquid has a predetermined ratio to the volume of the lumen of the catheter; and
a connector attached to container about the port, wherein the connector is configured to mate with the fitting to provide a sealed connection in which the cavity fluidly communicates with the lumen through the port;
affixing the device to the catheter by mating the connector with the fitting; and
applying positive pressure to the liquid to cause the liquid to exit the cavity through the port and enter the lumen of the catheter at the proximal end.

2. The method in accordance with claim 1, wherein the ratio is from about 1:2 to about 2:1.

3. The method in accordance with claim 1, wherein the liquid has a volume of from about 1 to about 5 milliliters.

4. The method in accordance with claim 1, wherein the cavity is essentially free from air.

5. The method in accordance with claim 1 wherein the catheter is a transcutaneous body catheter, wherein the distal end is internally positioned within a patient, and wherein the proximal end is external to the patient.

6. The method in accordance with claim 1, wherein the liquid is a catheter lock solution.

7. The method in accordance with claim 6, wherein the ratio is from about 0.8:1 to about 1:0.8.

8. The method in accordance with claim 6, wherein the catheter is a hemodialysis catheter.

9. The method in accordance with claim 8, further comprising:
allowing the device to remain affixed to the catheter until a hemodialysis treatment is indicated;
applying a negative pressure to the cavity to cause a predetermined volume of fluid to exit the lumen of the catheter at the proximal end and enter the cavity through the port;
disconnecting the device from the catheter; and
connecting one or more hemodialysis conduits to the catheter for the dialysis treatment.

10. The method in accordance with claim 1, wherein the liquid is a saline flush solution.

11. The method in accordance with claim 10, further comprising, prior to said affixing, introducing into the catheter a liquid selected from the group consisting of a dose of medicine and a dose of nutrients.

12. The method in accordance with claim 1, wherein the fitting is one of a male Luer coupling or a female Luer coupling, and the connector comprises the other of a male Luer coupling or a female Luer coupling, and wherein said affixing comprises press-fitting the fitting and the connector.

13. The method in accordance with claim 12, wherein the connector includes a fastener to maintain integrity between the fitting and the connector.

14. The method in accordance with claim 13, wherein the proximal end of the catheter defines a first flange or a first screw thread, and wherein the fastener defines a second flange or a second screw thread configured to cooperate with the first flange or first screw thread to maintain integrity between the fitting and the connector.

15. The method in accordance with claim 1, wherein the container comprises a disk shape.

16. The method in accordance with claim 1, wherein the container comprises a straight bellows configuration.

17. The method in accordance with claim 1, wherein the container comprises a fan-shaped bellows configuration.

18. The method in accordance with claim 1, wherein the port is a one-way valve.

19. The method in accordance with claim 1, wherein the port is a duck-bill valve.

20. The method in accordance with claim 1, wherein the port is a stopcock valve.

21. The method in accordance with claim 1, wherein the port is a manually actuated valve.

22. The method in accordance with claim 1, wherein the port is an orifice.

23. The method in accordance with claim 22, wherein the orifice has a diameter of from about 0.25 to about 2 mm.

24. The method in accordance with claim 22, wherein the orifice has a diameter of from about 0.75 to about 1 mm.

25. The method in accordance with claim 22, wherein the orifice has a diameter of about 0.5 mm.

26. A device for infusing a liquid into a catheter, the catheter defining lumen, a proximal end and a distal end, wherein the proximal end includes a fitting configured to mate with a connector, the device comprising:
a collapsible container defining a cavity and defining a port;
a liquid contained within the cavity, wherein the volume of the liquid has a predetermined ratio to the volume of the lumen of the catheter; and
a connector attached to container about the port, wherein the connector is configured to mate with the fitting to provide a sealed connection in which the cavity fluidly communicates with the lumen through the port;
wherein the port is configured to allow the passage of the liquid from the cavity into the lumen of the catheter at the proximal end when a positive pressure is applied to the liquid.

27. The device in accordance with claim 26, wherein the ratio is from about 1:2 to about 2:1.

28. The device in accordance with claim 26, wherein the liquid has a volume of from about 1 to about 5 milliliters.

29. The device in accordance with claim 26, wherein the cavity is essentially free from air.

30. The device in accordance with claim 26, wherein the liquid is a catheter lock solution.

31. The device in accordance with claim 30, wherein the ratio is from about 0.8:1 to about 1:0.8.

32. The device in accordance with claim 26, wherein the liquid is a saline flush solution.

33. The device in accordance with claim 26, wherein the connector comprises a male Luer coupling or a female Luer coupling configured to sealingly engage a corresponding fitting of a catheter.

34. The device in accordance with claim 33, wherein the connector includes a fastener to maintain integrity between the fitting and the connector.

35. The device in accordance with claim 34, wherein the fastener defines a first flange or a first screw thread configured to cooperate with a corresponding second flange or second screw thread of a catheter to maintain integrity between the fitting and the connector.

36. The device in accordance with claim 26, wherein the container comprises a disk shape.

37. The device in accordance with claim 26, wherein the container comprises a straight bellows configuration.

38. The device in accordance with claim 26, wherein the container comprises a fan-shaped bellows configuration.

39. The device in accordance with claim 26, wherein the port is a one-way valve.

40. The device in accordance with claim 26, wherein the port is a duck-bill valve.

41. The device in accordance with claim 26, wherein the port is a stopcock valve.

42. The device in accordance with claim 26, wherein the port is a manually actuated valve.

43. The device in accordance with claim 26, wherein the port is an orifice.

44. The device in accordance with claim 43, wherein the orifice has a diameter of from about 0.25 to about 2 mm.

45. The device in accordance with claim 43, wherein the orifice has a diameter of from about 0.75 to about 1 mm.

46. The device in accordance with claim 43, wherein the orifice has a diameter of about 0.5 mm.
